(19) United States
(12) Patent Application Publication
(10) Pub. No.: US 2002/0115115 A1
(43) Pub. Date: Aug. 22, 2002

(54) Uses of mammalian genes and related reagents

(76) Inventors: Kathleen M. Smith, Palo Alto, CA (US); Albert Zlotnik, Palo Alto, CA (US)

Correspondence Address: DNAX RESEARCH INSTITUTE, LEGAL DEPARTMENT, 901 CALIFORNIA AVENUE, PALO ALTO, CA, 94304, US

(21) Appl. No.: 09/920,318

(22) Filed: Jul. 31, 2001

Related U.S. Application Data
(60) Provisional application No. 60/222,258, filed on Aug. 1, 2000.

(51) Int. Cl. G01N 33/53

(52) U.S. Cl. 435/7.8; 424/130.1

(57) ABSTRACT

Methods for treating, diagnosing, or evaluating mucosal surface medical conditions. Correlations of chemokine or receptor expression with gastrointestinal status are provided.

This application claims benefit of U.S. Provisional Patent Application No. 60/222,258, filed Aug. 1, 2000.

### FIELD OF THE INVENTION

The present invention relates generally to uses of mammalian genes and related reagents. More specifically, the invention relates to identification of mammalian genes whose expression levels are implicated in medical conditions affecting the mucosal surfaces, e.g., inflammatory conditions. Diagnostic and therapeutic uses result.

### BACKGROUND OF THE INVENTION

Because inflammatory responses are often mediated by cytokine or chemokine activity, methods to evaluate synthesis of these signaling molecules would be advantageous for diagnosis of selected diseases. The present invention relates generally to identification of genes which may directly be of use to treat, or alternatively, to evaluate status of medical conditions affecting mucosal surfaces. See, e.g., Ogra, et al. (eds. 1999) Mucosal Immunity (2d ed.) Academic Press. The major mucosal surfaces include the gastrointestinal tract, the lungs and associated surfaces, and the female reproductive tract. Such mucosal surfaces include inflammatory bowel diseases such as ulcerative colitis and Crohn's Disease.

Inflammatory bowel disease (IBD) refers to a group of gastrointestinal disorders characterized by a chronic non-specific inflammation of portions of the gastrointestinal tract. Ulcerative colitis and Crohn's Disease are the most prominent examples of IBD in humans. They are associated with many symptoms and complications, including growth retardation in children, rectal prolapse, blood in stools (e.g., melena and/or hematochezia), wasting, iron deficiency, and anemia, e.g. iron deficiency anemia and anemia of chronic disease or of chronic inflammation. The etiology or etiologies of IBD are unclear. See, Wyngaarden and Smith (eds.) Cecil's Textbook of Medicine (W. B. Saunders Co. 1985), Berkow (ed.) The Merck Manual of Diagnosis and Therapy (Merck Sharp & Dohme Research Laboratories, 1982), and Harrison's Principles of Internal Medicine, 12th Ed., McGraw-Hill, Inc. (1991), all of which are incorporated herein by reference.

Ulcerative colitis refers to a chronic, non-specific, inflammatory, and ulcerative disease having manifestations primarily in the colonic mucosa. It is frequently characterized by bloody diarrhea, abdominal cramps, blood and mucus in the stools, malaise, fever, anemia, anorexia, weight loss, leukocytosis, hypoalbuminemia, and an elevated erythrocyte sedimentation rate (ESR). Complications can include hemorrhage, toxic colitis, toxic megacolon, occasional rectovaginal fistulas, and an increased risk for the development of colon cancer.

Ulcerative colitis is also associated with complications distant from the colon, such as arthritis, ankylosing spondylitis, sacroileitis, posterior uveitis, erythema nodosum, pyoderma gangrenosum, and episcleritis. Treatment varies considerably with the severity and duration of the disease. For instance, fluid therapy to prevent dehydration and electrolyte imbalance is frequently indicated in a severe attack. Additionally, special dietary measures are sometimes useful. Medications include various corticosteroids, sulphasalazine and some of its derivatives, and possibly immunosuppressive drugs.

Crohn's Disease shares many features in common with ulcerative colitis. Crohn's Disease is distinguishable in that lesions tend to be sharply demarcated from adjacent normal bowel, in contrast to the lesions of ulcerative colitis which are fairly diffuse. Additionally, Crohn's Disease predominately afflicts the ileum (ileitis) and the ileum and colon (ileocolitis). In some cases, the colon alone is diseased (granulomatous colitis) and sometimes the entire small bowel is involved (jejunoileitis). In rare cases, the stomach, duodenum, or esophagus are involved. Lesions include a sarcoid-type epithelioid granuloma in roughly half of the clinical cases. Lesions of Crohn's Disease can be transmural including deep ulceration, edema, and fibrosis, which can lead to obstruction and fistula formation as well as abscess formation. This contrasts with ulcerative colitis which usually yields much shallower lesions, although occasionally the complications of fibrosis, obstruction, fistula formation, and abscesses are seen in ulcerative colitis as well.

Treatment is similar for these diseases and includes steroids, sulphasalazine and its derivatives, and immunosuppressive drugs such as cyclosporin A, mercaptopurine and azathioprine.

The severe complications of problems with mucosal surfaces can be seriously debilitating, and eventually may lead to death. Thus, a need exists for effective treatment, both prophylactic and curative, to alleviate the symptoms of those conditions. Alternatively, methods of diagnosis, e.g., of abnormal or modified health of those tissues will be useful. The present invention provides both.

### SUMMARY OF THE INVENTION

The present invention provides methods of diagnosing or evaluating a mucosal condition comprising evaluating expression of: a chemokine selected from TARC or MIP-3α; or a chemokine receptor selected from CCR4, CCR6, CCR7, or CCR8. Preferably, the evaluating is a plurality of MIP-3α, TARC, CCR7, or CCR8; or the mucosa is selected from: a gastrointestinal mucosa; a pulmonary mucosa; or a female reproductive tract mucosa.

In another embodiment, the invention provides methods of treating an inflammatory bowel disease comprising blocking signaling: mediated by a chemokine selected from: TARC or MIP-3α; or effected by a chemokine receptor selected from: CCR4; CCR6; CCR7; or CCR8. In certain embodiments, the bowel disease is ulcerative colitis or Crohn's disease; or the treating is preventing appearance of symptoms. Preferably, the blocking is of signaling: mediated by TARC; or effected by a chemokine receptor selected from: CCR4; or CCR7. In another embodiment, the blocking is by administering an antibody raised against the chemokine or the chemokine receptor.

### DETAILED DESCRIPTION OF THE PREFERRED EMBODIMENTS

I. General

The mucosal surfaces, e.g., the gastrointestinal, respiratory, and female reproductive tracts, share similarities in structure and function. Inflammatory and other processes which affect them are likely to involve the chemokine signaling mechanisms.

The gastrointestinal system is the portal through which nutritive substances, vitamins, minerals, and fluids enter the body. The gut surface is significant in being topologically “external” to the organism, which makes it a significant surface which contacts the environment. This boundary serves as the initial barrier through which many foreign agents or organisms must pass to enter the body. Typically, there are three layers of smooth muscle. The wall is lined by mucosa and is covered by serosa. The serosa continues onto the mesentery, which contains nerves, lymphatic, and blood vessels supplying the tract. See, e.g., Yamada (1999) Textbook of Gastroenterology; and Spiro (1993) Clinical Gastroenterology.

The present invention resulted from studies directed to whether modified expression of chemokines or chemokine receptors correlated with conditions affecting the mucosal surfaces. In particular, gastrointestinal inflammations include, e.g., inflammatory bowel disease (IBD), which are represented by two primary types, e.g., Crohn's disease or ulcerative colitis. Increased expression of chemokines could result in recruitment of inflammatory cells, e.g., macrophages, dendritic cells, or lymphocytes, and which may contribute to lesion development in IBD and related conditions. A set of genes with no known disease associations in gastrointestinal conditions were selected to analyze on a cDNA panel. These included several chemokines and chemokine receptors. In addition, several chemokines known to be up-regulated in IBD and their receptors, were also analyzed on the cDNA panel as positive controls.

II. Antagonists

Blockage of the signaling pathway can be achieved by antagonists of the chemokine, e.g., antibodies to the ligand, antibodies to the receptor, etc. Interference with the ligand-receptor interaction has proven to be an effective strategy for the development of antagonists.

There are various means to antagonize the signaling mediated by ligand. Two apparent means are to block the ligand with antibodies; a second is to block the receptor with antibodies. Various epitopes should exist on each which will block their interaction, e.g., causing steric hindrance blocking interaction. The correlation of ability to block signaling would not necessarily be expected to correlate with binding affinity to either ligand or receptors.

Alternatively, small molecule libraries may be screened for compounds which may block the interaction or signaling mediated by an identified ligand-receptor pairing.

The present invention provides for the use of an antibody or binding composition which specifically binds to a specified chemokine ligand, preferably mammalian, e.g., primate, human, cat, dog, rat, or mouse. Antibodies can be raised to various chemokine proteins, including individual, polymorphic, allelic, strain, or species variants, and fragments thereof, both in their naturally occurring (full-length) forms or in their recombinant forms. Additionally, antibodies can be raised to receptor proteins in both their native (or active) forms or in their inactive, e.g., denatured, forms. Anti-idiotypic antibodies may also be used.

A number of immunogens may be selected to produce antibodies specifically reactive with ligand or receptor proteins. Recombinant protein is a preferred immunogen for the production of monoclonal or polyclonal antibodies. Naturally occurring protein, from appropriate sources, e.g., primate, rodent, etc., may also be used either in pure or impure form. Synthetic peptides, made using the appropriate protein sequences, may also be used as an immunogen for the production of antibodies. Recombinant protein can be expressed and purified in eukaryotic or prokaryotic cells as described, e.g., in Coligan, et al. (eds. 1995 and periodic supplements) Current Protocols in Protein Science John Wiley & Sons, New York, N.Y.; and Ausubel, et al (eds. 1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be done if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of ligand or receptor proteins can be raised by immunization of animals with conjugates of the fragments with carrier proteins. Monoclonal antibodies are prepared from cells secreting the desired antibody. These antibodies can be screened for binding to normal or defective protein. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567; and Queen, et al. (1989) Proc. Nat'l Acad. Sci. USA 86:10029-10033; or made in transgenic mice, see Mendez, et al. (1997) Nature Genetics 15:146-156; also see Abgenix and Medarex technologies.

Antibodies are merely one form of specific binding compositions. Other binding compositions, which will often have similar uses, include molecules that bind with specificity to ligand or receptor, e.g., in a binding partner-binding partner fashion, an antibody-antigen interaction, or in a natural physiologically relevant protein-protein interaction, either covalent or non-covalent, e.g., proteins which specifically associate with desired protein. The molecule may be a polymer, or chemical reagent. A functional analog may be a protein with structural modifications, or may be a structurally unrelated molecule, e.g., which has a molecular shape which interacts with the appropriate binding determinants. Antibody binding compounds, including binding fragments, of this invention can have significant diagnostic or therapeutic value. They can be useful as non-neutralizing binding compounds and can be coupled to toxins or radionuclides so that when the binding compound binds to the antigen, a cell expressing it, e.g., on its surface, is killed. Further, these binding compounds can be conjugated to drugs or other therapeutic agents, either directly or indirectly by means of a linker, and may effect drug targeting.

III. Diagnostic Uses; Therapeutic Compositions, Methods

The inflammatory bowel diseases of the gut encompassing Crohn's disease and ulcerative colitis are complex chronic diseases, with symptoms of inflammation. The etiology and pathogenesis are poorly understood, but they cause significant morbidity in many patients. These diseases are frequently relapsing diseases ultimately leading to destruction of mucosal tissue. Recent evidence suggests that a pathological activation of the mucosal immune system in response to antigens is a key factor in the pathogenesis of IBD. Furthermore, changes in cell migration and chemokine production appear to correlate with the perpetuation of IBD and the postoperative recurrence of Crohn's disease.

Collectively these studies suggest that antagonizing these chemokines or their receptors, with the appropriate entity may offer a therapeutic modality in mucosal conditions or diseases, e.g., inflammatory conditions of the alimentary canal, respiratory mucosa, or female reproductive tract. Alimentary canal medical conditions or diseases include, e.g., gingivitis, periodontal disease, gastritis, ileitis, colitis, IBD, ulcers, and gastrointestinal tumors or lymphomas, e.g., gastric carcinoma, gastric lymphoma, colon carcinoma, rectal carcinoma, colorectal lymphoma, and lymphoma of the small intestine. See, e.g., Yamada (1999) Textbook of Gastroenterology; and Spiro (1993) Clinical Gastroenterology. Respiratory tract conditions include, e.g., bronchitis, alveolitis, emphysema, asthma, interstitial lung diseases, chronic obstructive pulmonary disease (COPD; see, e.g., Murray (1996) Frontline Treatment of COPD), cancers, small cell and non small cell lung cancers, sarcomas, lymphomas, etc. See, e.g., Albert, et al. (1999) Comprehensive Respiratory Medicine Mosby; Fishman and Elias (eds. 1998) Fishman's Pulmonary Diseases and Disorders McGraw Hill; and Kradin (ed. 1996) Immunopathology of Lung Disease Butterworth-Heinemann. Female reproductive tract conditions include vaginitis, cervicitis, uteritis, fallopitis, and various oncological conditions, e.g., cervical carcinoma, endometrial carcinoma, and ovarian carcinoma. See, e.g., Anderson and Symmers (eds. 1991) Female Reproductive System (Systemic Pathology, Vol 6).

Diagnostic methods include such aspects as prediction of prognosis; definition of subsets of patients who will either respond or not respond to a particular therapeutic course; diagnosis of mucosal diseases or subtypes of conditions or diseases; or assessing response to therapy. For example, subtypes of inflammatory diseases my be defined molecularly by the comparative expression levels of TARC, MDC, MIP-3α, MIP-3β, I-309, CCR4, CCR6, CCR7, CCR8, or various combinations thereof.

Antagonists to chemokine mediated signaling have been implicated in a manner suggesting significant therapeutic effects, e.g., to decrease or prevent occurrence of symptoms. Small molecule antagonists for 7 transmembrane receptors and chemokine receptors are well known. Pertussis toxin can block the interaction of such receptors with the associated signaling G-protein coupled receptors.

The antagonists of the present invention can be administered alone or in combination with another inhibitor of the same or accompanying pathway; or other compounds used for the treatment of symptoms, e.g., antagonists, or steroids such as glucocorticoids.

In contrast, chemokines could be administered as an adjuvant for immunization, e.g., to trigger mucosal immunity, e.g., papilloma virus vaccine for the reproductive tract. See, e.g., Ogra, et al. (eds. 1999) Mucosal Immunity (2d ed.) Academic Press.

To prepare pharmaceutical or sterile compositions including the antibody or binding composition thereof, the antibody or binding composition is admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984).

Antibodies or binding compositions are normally administered parentally, preferably intravenously. Since such protein or peptide antagonists may be immunogenic they are preferably administered slowly, either by a conventional IV administration set or from a subcutaneous depot, e.g. as taught by Tomasi, et al, U.S. Pat. No. 4,732,863.

When administered parenterally the antibodies or fragments will be formulated in a unit dosage injectable form (solution, suspension, emulsion) in association with a pharmaceutically acceptable parenteral vehicle. Such vehicles are inherently nontoxic and nontherapeutic. The antagonist may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ) or intramuscular (IM) injection. The proportion of antagonist and additive can be varied over a broad range so long as both are present in effective amounts. The antibody is preferably formulated in purified form substantially free of aggregates, other proteins, endotoxins, and the like, at concentrations of about 5 to 30 mg/ml, preferably 10 to 20 mg/ml. Preferably, the endotoxin levels are less than 2.5 EU/ml. See, e.g., Avis, et al. (eds.)(1993) Pharmaceutical Dosage Forms: Parenteral Medications 2d ed., Dekker, N.Y.; Lieberman, et al. (eds. 1990) Pharmaceutical Dosage Forms: Tablets 2d ed., Dekker, N.Y.; Lieberman, et al. (eds. 1990) Pharmaceutical Dosage Forms: Disperse Systems Dekker, N.Y.; Fodor, et al. (1991) Science 251:767-773, Coligan (ed.) Current Protocols in Immunology; Hood, et al. Immunology Benjamin/Cummings; Paul (ed.) Fundamental Immunology; Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York.

Selecting an administration regimen for an antagonist depends on several factors, including the serum or tissue turnover rate of the antagonist, the level of symptoms, the immunogenicity of the antagonist, and the accessibility of the target cells. Preferably, an administration regimen maximizes the amount of antagonist delivered to the patient consistent with an acceptable level of side effects. Accordingly, the amount of antagonist delivered depends in part on the particular antagonist and the severity of the condition being treated. Guidance in selecting appropriate doses is found in the literature on therapeutic uses of antibodies, e.g. Bach et al., chapter 22, in Ferrone, et al. (eds. 1985) Handbook of Monoclonal Antibodies Noges Publications, Park Ridge, N.J.; and Haber, et al. (eds.) (1977) Antibodies in Human Diagnosis and Therapy, Raven Press, New York, N.Y. (Russell, pgs. 303-357, and Smith, et al., pgs. 365-389).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of the inflammation, e.g., level of inflammatory cytokines produced. Preferably, an antibody or binding composition thereof that will be used is derived from the same species as the animal targeted for treatment, thereby minimizing a humoral response to the reagent.

The total weekly dose ranges for antibodies or fragments thereof, which specifically bind to ligand or receptor range generally from about 10 μg, more generally from about 100 μg, typically from about 500 μg, more typically from about 1000 μg, preferably from about 5 mg, and more preferably from about 10 mg per kilogram body weight. Generally the range will be less than 100 mg, preferably less than about 50 mg, and more preferably less than about 25 mg per kilogram body weight.

The weekly dose ranges for antagonists of chemokine receptor mediated signaling, e.g., antibody or binding fragments, range from about 1 μg, preferably at least about 5 μg, and more preferably at least about 10 μg per kilogram of body weight. Generally, the range will be less than about 1000 μg, preferably less than about 500 μg, and more preferably less than about 100 μg per kilogram of body weight. Dosages are on a schedule which effects the desired treatment and can be periodic over shorter or longer term. In general, ranges will be from at least about 10 μg to about 50 mg, preferably about 100 μg to about 10 mg per kilogram body weight. Small molecule antagonists will typically have similar molar concentrations, but because they have smaller molecular weights, will have lesser weight doses.

The present invention also provides for administration of antibodies or binding compositions in combination with known therapies, e.g., steroids, particularly glucocorticoids, which alleviate the symptoms, e.g., associated with inflammation, or antibiotics or anti-infectives. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day. Suitable dose combinations with antibiotics, anti-infectives, or anti-inflammatories are also known.

The phrase “effective amount” means an amount sufficient to ameliorate a symptom or sign of the medical condition. Typical mammalian hosts will include mice, rats, cats, dogs, and primates, including humans. An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone An effective amount of antagonist will decrease the symptoms typically by at least about 10%; usually by at least about 20%; preferably at least about 30%; or more preferably at least about 50%. The present invention provides reagents which will find use in therapeutic applications as described elsewhere herein, e.g., in the general description for treating disorders associated with the indications described, e.g., inflammatory conditions, chronic or acute, etc. See, e.g., Dayer (1999) J. Clin. Invest. 104:1337-1339; Gracie, et al. (1999) J. Clin. Invest. 104:1393-1401; Berkow (ed.) The Merck Manual of Diagnosis and Therapy, Merck & Co., Rahway, N.J.; Thorn, et al. Harrison's Principles of Internal Medicine, McGraw-Hill, NY; Gilman, et al. (eds.) (1990) Goodman and Gilman's: The Pharmacological Bases of Therapeutics, 8th Ed., Pergamon Press; Remington's Pharmaceutical Sciences, 17th ed. (1990), Mack Publishing Co., Easton, Penn; Langer (1990) Science 249:1527-1533; Merck Index, Merck & Co., Rahway, N.J.; and Physician's Desk Reference (PDR).

The broad scope of this invention is best understood with reference to the following examples, which are not intended to limit the inventions to the specific embodiments.

### EXAMPLES

I. General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Meth. Enzymol., vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering. Principle and Methods 12:87-98, Plenum Press, N.Y.; and Crowe, et al. (1992) QIAexpress: The High Level Expression & Protein Purification System QIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank and others.

II. RNA Preparation

RNA was extracted from intestinal tissue either using guanidinium thiocyanate followed by centrifugation in cesium chloride for surgical samples or RNA STAT-60 (Tel-Test) for biopsies. RNA quality was assessed by agarose gel electrophoresis. Five μg of total RNA was treated with RNAse-free DNAse I (Boehringer Mannheim) in First Strand Synthesis Buffer in the presence of RNAsin (Promega). Samples were incubated for 20 minutes at 37° C., heated for 10 minutes at 70° C., and then immediately chilled on ice. A mixture of 2.5 μg of oligo d(T)₁₂₋₁₅ (Boehringer Mannheim) and 250 ng of random hexamers (Promega) was added to each sample. Samples were heated to 70° C. for 10 minutes, rapidly chilled on ice, and then briefly spun in a microfuge. cDNA was generated from the RNA using Superscript II reverse transcriptase (GIBCO-BRL) according to manufacturer's instructions in a final volume of 100 μL.

III. Patient Evaluation

Patient intestinal samples were grouped two different ways. In the first grouping patient samples were into one of five groups, based on the clinical and pathological diagnoses of the patients. Non-lesional samples from the same patients were also studied, however, none of these samples was statistically significant from the control group.

A. Controls: There were twenty-eight samples in the control group, including samples from both colon and small intestine. Samples in this group were surgical samples from patients with normal adjacent intestinal tissue removed during excision of a tumor (n=12), biopsies from patients with chronic colitis (n=4), surgical specimens from patients diagnosed with diverticulosis (n=5), and biopsies from patients undergoing routine endoscopy (n=2). In addition, five intestinal samples were collected during autopsy.

B. Crohn's Diseas, lesional, no steroids: There were six samples from patients, diagnosed with Crohn's Disease who had no history of recent steroid use. Four of these were biopsies and two surgical samples.

C. Crohn's Disease, lesional, steroids: There were four samples from patients, diagnosed with Crohn's Disease who had recently been treated with steroids. All four were surgical samples.

D. Ulcerative colitis, lesional, no steroids: There were eight samples from patients, diagnosed with ulcerative colitis who had no history of recent steroid use. Seven of these were biopsies and one a surgical sample.

E. Ulcartive colitis, lesional, steroids: There are five samples from patients, diagnosed with ulcerative colitis and who had recently been treated with steroids. All five were surgical samples.

In addition to the above groups the data collected from the above samples were all grouped as described below and analyzed as described in the next section.

A. Controls: This group was the same as described above.

B. Crohn's Disease, lesional, all: This group had 15 samples and included all samples in both Crohn's Disease lesional groups described above as well as five additional samples for which treatment history was not available.

C. Ulcartive colitis, lesional, all: This group had 22 samples and included all samples in both Ulcerative colitis lesional groups described.

IV. Gene Expression Analysis by TaqMan

Ten ng of cDNA/reaction was analyzed for expression of selected chemokines, chemokine receptors, and ubiquitin on a GeneAmp 5700 Sequence Detector (PE Applied Biosystems) in a 25 μL reaction. Expression of chemokines and chemokine receptors was detected using primers and probe (PE Applied Biosystems) with TaqMan Universal Master Mix (PE Applied Biosystems) or primers alone and SYBR Green PCR Master Mix (PE Applied Biosystems). TaqMan reagents to detect expression of given chemokines or their receptors were extensively tested to verify that they only recognized the appropriate target sequence and not closely related family members. Ubiquitin was detected using 200 nM primers (F: CACTTGGTCCTGCGCTTGA; R: CAATTGGGAATGCAACAACTTTAT) with SYBR Green PCR Mater Mix. The data was analyzed to calculate a cycle threshold value (Ct) for each sample with GeneAmp 5700 SDS Software (PE Applied Biosystems). The relative level of CCL18 mRNA in the tissue using the following formula: 1.8(Ct of ubiquitin −Ct of gene of interest)10,000 for each sample. Mean and standard error were calculated for each group.

V. Statistical analysis

Statistical analysis was performed with JMP 3.2.2 (SAS Institute, Inc.). The TaqMan data for the relative level of chemokine and chemokine recpetors was log transformed and a one-way ANOVA performed. The log transformed of chemokine and chemokine recpetor level was used as the dependent variable and the disease group was used as the independent variable. All groups were compared to the control group using a modified Student t-test, Dunnett's method.

Increased expression of several chemokine and their receptors in lesional tissue samples from patients with inflammatory bowel diseases, Crohn's Disease and ulcerative colitis was detected (see Table 1).

TABLE 1
Gene expression levels of chemokines and receptors.
Bold type indicates a statistically significant fold
increase in gene expression over that of the control group.
Fold Increase in Gene Expression over Controls

| | n | TARC | CCR4 | CCR8 | MIP-3α | CCR6 | CCR7 | TNF-α |
|---|---|---|---|---|---|---|---|---|
| Controls | 28 | 1.0 | 1.0 | 1.0 | 1.0 | 1.0 | 1.0 | 1.0 |
| Crohn's Lesional, | 6 | 3.8 | 7.9 | 8.3 | 7.4 | 6.2 | 3.9 | 4.4 |
| no steriods | | | | | | | | |
| Crohn's Lesional, | 4 | 1.6 | 1.8 | 0.9 | 2.5 | 0.9 | 2.3 | 0.5 |
| steriods | | | | | | | | |
| Crohn's Lesional, | 15 | 2.9 | 4.4 | 3.5 | 2.4 | 2.5 | 4.2 | 1.6 |
| all | | | | | | | | |
| Ulcerative colitis, | 8 | 2.7 | 10.0 | 7.5 | 5.0 | 2.5 | 3.1 | 2.4 |
| no steriods | | | | | | | | |
| Ulcerative colitis, | 5 | 2.4 | 3.3 | 1.2 | 1.6 | 0.8 | 2.0 | 0.9 |
| steriods | | | | | | | | |
| Ulcerative colitis, | 22 | 2.4 | 5.2 | 2.2 | 2.4 | 1.2 | 2.7 | 1.4 |
| all | | | | | | | | |

The chemokine receptors studied here are expressed on lymphocytes and antigen expressing cells. Recruitment of these cell types, which promote immune responses, into intestinal lesions may result in the maintenance and magnification of the chronic underlying these debilitating diseases. While the expression of several of the chemokines and chemokine receptors studied here is decreased by steroid treatment, it is not abolished in all cases. The results above indicate that these molecules may represent new targets for dampening the immune response, without the broad systemic effects induced by chronic steroid use.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of diagnosing or evaluating a mucosal condition, said method comprising evaluating expression of:
a) a chemokine selected from TARC or MIP-3α or
b) a chemokine receptor selected from CCR4, CCR6, CCR7, or CCR8.

2. The method of claim 1, wherein said evaluating is a plurality of MIP-3α, TARC, CCR7, or CCR8.

3. The method of claim 1, wherein said mucosa is selected from:
a) a gastrointestinal mucosa;
b) a pulmonary mucosa; or
c) a female reproductive tract mucosa.

4. A method of treating an inflammatory bowel disease, said method comprising blocking signaling:
a) mediated by a chemokine selected from:
i) TARC; or
ii) MIP-3α; or
b) effected by a chemokine receptor selected from:
i) CCR4;
ii) CCR6;
iii) CCR7; or
iv) CCR8.

5. The method of claim 4, wherein:
a) said bowel disease is ulcerative colitis or Crohn's disease; or
b) said treating is preventing appearance of symptoms.

6. The method of claim 4, blocking signaling:
a) mediated by TARC; or
b) effected by a chemokine receptor selected from:
i) CCR4; or
iii) CCR7.

7. The method of claim 5, wherein said blocking is by administering an antibody raised against said chemokine or said chemokine receptor.
